(19) United States
(12) Patent Application Publication
(10) Pub. No.: US 2010/0136070 A1
(43) Pub. Date: Jun. 3, 2010

(54) METHODS, DEVICES, AND COMPOSITIONS FOR DERMAL FILLING

(75) Inventors: John Daniel Dobak, La Jolla, CA (US); Glenn Downes Prestwich, Eastsound, WA (US)

Correspondence Address: WILSON, SONSINI, GOODRICH & ROSATI, 650 PAGE MILL ROAD, PALO ALTO, CA, 94304-1050, US

(73) Assignee: JAKK Group, Inc., La Jolla, CA (US)

(21) Appl. No.: 12/630,733

(22) Filed: Dec. 3, 2009

Related U.S. Application Data
(60) Provisional application No. 61/119,541, filed on Dec. 3, 2008.

(51) Int. Cl. A61K 8/02; A61K 8/73; A61K 8/11; A61K 8/81; A61K 8/65; A61Q 19/08; A61K 8/24; C07H 1/00; C08F 120/18; C07K 14/78; C08B 1/00; C08B 37/02

(52) U.S. Cl. 424/401; 514/54; 514/59; 424/78.03; 514/12; 514/57; 424/602; 536/123.1; 526/329.7; 530/356; 536/56; 536/112

(57) ABSTRACT

Provided herein are methods, devices, and compositions for dermal filling. Also described herein are dermal filler cross-linked compositions and methods for making such compositions. Such compositions comprise, for example, a cross-linked composition of hyaluronic acid, derivatives of hyaluronic acid or mixtures thereof, alginic acid, derivatives of hyaluronic acid or mixtures thereof and calcium ions.

### RELATED APPLICATIONS

This application claims the benefit of U.S. Provisional Patent Application No. 61/119,541 filed Dec. 3, 2008, the contents of which are incorporated by reference in its entirety.

### BACKGROUND OF THE INVENTION

The use of nonsurgical products and devices to correct facial contour defects and signs of skin aging (e.g. wrinkles or rhytids) has expanded.

### SUMMARY OF THE INVENTION

Presented herein are methods for the delivery of dermal filler material for providing volume for skin contouring, skin correction or modification, such as for example, skin wrinkles and surface defect correction.

In one aspect is a method for correcting or modifying skin defects comprising:
providing substantially or partially dry microparticles of dermal filler; and
delivering the substantially or partially dry microparticles to the dermis wherein the volume of the dermis is increased.

In one embodiment is a method for correcting or modifying skin defects wherein the dermal filler comprises a material selected from hyaluronic acid, dextran, polymethacrylate, agarose, collagen, hydroxyapatite, polymethylmethacrylate, and carboxymethyl cellulose. In another embodiment the dermal filler comprises hyaluronic acid. In a further embodiment the hyaluronic acid is substantially cross-linked. In yet another embodiment the dermal filler comprises dextran. In a further embodiment dextran is substantially cross-linked. In one embodiment delivering the substantially or partially dry microparticles of dermal filler to the dermis comprises accelerating the substantially or partially dry microparticles at a velocity sufficient to penetrate the skin.

In another aspect is a method for delivering a dermal filler comprising delivering at a high velocity substantially or partially dry microparticles of dermal filler through the skin and into the dermis using a substantially dry powder needleless injection device.

In one embodiment is a method for delivering a dermal filler wherein the substantially or partially dry microparticles of dermal filler comprises a material selected from hyaluronic acid, dextran, polymethacrylate, agarose, collagen, hydroxyapatite, polymethylmethacrylate, and carboxymethyl cellulose. In another embodiment the dermal filler comprises hyaluronic acid. In yet another embodiment hyaluronic acid is substantially cross-linked. In a further embodiment the dermal filler comprises dextran. In yet a further embodiment dextran is substantially cross-linked. In another embodiment the substantially or partially dry microparticles further comprises or are coated with an anesthetic agent. In yet another embodiment the anesthetic agent is lidocaine. In one embodiment the substantially or partially dry microparticles further comprise or are coated with an anti-inflammatory agent. In another embodiment the substantially or partially dry microparticles further comprise or are coated with dermal growth factors or fibroblast growth factors, including but not limited to proteins, steroids, cytokines, or hormones. In a further embodiment the substantially or partially dry microparticles further comprise or are coated with a paralytic. In yet a further embodiment the paralytic is clostridium botulinum toxin. In another embodiment the substantially or partially dry microparticles are coated with a biocompatible material having a sufficient high surface hardness wherein the penetration properties of the microparticles are enhanced. In one embodiment is a method for providing volume for skin contouring, skin defect correction and surface defect correction comprising administering a dermal filler composition described herein. In a further embodiment is a method for contouring facial features comprising administering a dermal filler composition described herein. In another embodiment is a method for reducing the pain associated with needle delivery devices comprising administering a dermal filler composition described herein via a needless injection device. In another embodiment is a method of treating broader dermal areas which are difficult to access using a needle based delivery device comprising administering a dermal filler described herein. In one embodiment is a method for providing a more uniform and controlled delivery of a dermal filler described herein to the dermis of the skin. In yet another embodiment is a method for delivering a dermal filler described herein in a unique coverage area shape, such as but not limited to, periorbital crescents, acne scars, lip features comprising administering a dermal filler using custom nozzle shapes and designs. In yet another embodiment is a method for treating patient discomfort and pain for patients undergoing dermal filling procedures.

In a further embodiment is a method of delivering substantially or partially dry microparticles comprising a hydrophilic dermal filler agent, such as by way of example only, hyaluronic acid or carboxymethyl cellulose) uniformly to the dermis of the skin thereby allowing the microparticles to swell by absorbing moisture from the surrounding tissue to provide a volumizing effect. In one embodiment, the volumizing effect corrects or modifies skin surface defects, wrinkles, adds volume or the like.

Also presented herein is a dermal filler composition comprising a substantially or partially dry microparticle comprising a material selected from hyaluronic acid, dextran, polymethacrylate, agarose, collagen, hydroxyapatite, polymethylmethacrylate, and carboxymethyl cellulose.

In one embodiment is a dermal filler composition wherein the material comprises hyaluronic acid. In another embodiment is a dermal filler composition wherein the material is hyaluronic acid. In a further embodiment is a dermal filler composition wherein the hyaluronic acid is substantially cross-linked. In yet another embodiment is a dermal filler composition wherein the material comprises dextran. In one embodiment is a dermal filler composition wherein the material is dextran. In another embodiment, the dextran is substantially cross-linked. In yet another embodiment is a dermal filler composition further comprising a pharmaceutically acceptable carrier. In one embodiment the dermal filler composition is substantially free of a pharmaceutically acceptable carrier.

### BRIEF DESCRIPTION OF THE DRAWINGS

The features of the embodiments described herein are set forth with particularity in the appended claims. A better understanding of the features and advantages presently described herein will be obtained by reference to the following detailed description that sets forth illustrative embodiments, in which the principles are utilized, and the accompanying drawings of which:

FIG. 1 shows the first step in a process of making a dermal filler described herein.

FIG. 2 shows the second step in a process of making a dermal filler described herein.

### DETAILED DESCRIPTION OF THE INVENTION

Injectable devices currently used for soft tissue augmentation may be categorized by both substance and source. They include the 2 biologic fillers: collagen derived from bovine or human sources and hyaluronic acid (HA) derived from avian, mammalian, or bacterial sources. Synthetic fillers include poly-1-lactic acid, dextran, hydroxylapatite microparticles, carboxymethyl cellulose polyethylene oxide hydrogel, and the permanent, recently approved combination of polymethylmethacrylate and bovine collagen.

Techniques for the correction of nasolabial folds, forehead and glabellar wrinkles, marrionette lines, smoker's lines, and lip augmentation have been developed. Temporary, biocompatible agents not requiring allergic skin testing prior to administration are desired.

Hyaluronic Acids (HA) are a naturally occurring linear polysaccharide found in the extracellular matrix of connective tissue, synovial fluid, and other tissues of all animal species. In humans, it serves as the ground substance of the dermis and fascia and is an important component of most fluid mediums because of its viscoelastic properties. Its physical functions include space filling, lubrication, shock absorption, and protein exclusion. Its biochemical roles include the modulation of inflammatory cells, interaction with the proteoglycans of the extracellular matrix, and assisting in free radical scavenging. Importantly, HA has no species or tissue-specific antigenic properties, and thus has no innate potential for allergic or immunogenic reaction in humans, regardless of its source.

In some embodiments, the dermal fillers described herein are injected into the upper dermis, mid dermis, deep dermis and subcutaneous tissue. In further embodiments, upper and mid dermal injections are used to correct fine facial lines or skin surface defects, while deeper dermal injections are used to correct more significant skin defects such as wrinkles and scars. In further embodiments, subcutaneous injections are used to increase volume in the face and other areas of the body, such as breasts, or to correct deeper scars.

Dermal filler injection tunneling techniques are tedious for the physician as the filler must be deposited directly beneath each area or wrinkle being treated. The procedure is highly user (physician, physician assistant, etc.) dependent and the user can have significant difficulty targeting the desired layers of tissue. The volume (milligrams of filler) of filler delivered to a particular region may be highly variable and dependent on the ability of the physician to control the syringe/plunger extrusion force delivery and estimate the volume administered. The administered filler (e.g. hyaluronic acid) may be delivered in globules that can cause unevenness or lumpiness in the tissue. There is no suitable way to enhance the volume of the dermis over a region to correct thinning of the dermis secondary to aging and sun exposure. In some embodiments, cross-linking of HA is used to increase the longevity of the filler in the dermis. When small gauge needles are used the degree of cross-linking is limited by the extrusion force required to deliver higher cross-linking forms often resulting in tissue trauma. In addition, it is difficult to provide and maintain uniform particle distribution throughout the syringe and in the extruded filler blend when using a syringe to deliver particle based dermal fillers. Last, the volume displacement caused by the filler and multiple needle insertions are often painful to the patient. These syringe injection procedures are not acceptable to patients with needle phobia.

### Dermal Filler Substantially or Partially Dry Microparticles

Presented herein is a dermal filler composition comprising a substantially or partially dry microparticle comprising a material selected from hyaluronic acid, dextran, polymethacrylate, agarose, collagen, hydroxyapatite, polymethylmethacrylate, and carboxymethyl cellulose.

In one embodiment is a dermal filler composition wherein the substantially or partially dry material comprises hyaluronic acid. In another embodiment is a dermal filler composition wherein the substantially or partially dry material is hyaluronic acid. In a further embodiment is a dermal filler composition wherein the hyaluronic acid is substantially cross-linked. In yet another embodiment is a dermal filler composition wherein the substantially or partially dry material comprises dextran. In one embodiment is a dermal filler composition wherein the substantially or partially dry material is dextran. In another embodiment, the dextran is substantially cross-linked. In yet another embodiment is a dermal filler composition further comprising a pharmaceutically acceptable carrier. In one embodiment the dermal filler composition is substantially free of a pharmaceutically acceptable carrier. In another embodiment is a dermal filler composition further comprising an anesthetic agent, such as by way of example only, lidocaine. In another embodiment is a dermal filler composition further comprising an anti-inflammatory agent. In yet another embodiment is a dermal filler composition further comprising a dermal growth factor or fibroblast growth factor, including but not limited to proteins, steroids, cytokines, or hormones. In yet another embodiment is a dermal filler composition further comprising a paralytic, such as by way of example only, clostridium botulinum toxin.

In some embodiments, biocompatible polymers and materials suitable for dermal filler use are produced as microparticles of appropriate size, surface hardness, and density for administration by substantially dry powder injection. Examples include but are not limited to: hyaluronic acid, cellulose and its derivatives such as carboxymethyl cellulose, agarose or alginate hydrogels, dextrans, poly lactic or lactic galactic polymers, polymethylmethacrylate, and hydroxyapatite. In other embodiments, collagen and other extracellular matrix proteins formulated as dry microparticles are used for substantially dry powder injection dermal filling.

In other embodiments, hyaluronic acid is the dermal filling agent. In further embodiments, HA skin fillers approved by the FDA in the US are used such as by way of example only, Juvederm (Ultra and Ultra Plus), Hylaform, Captique, Restylane, Hydrelle, Prevelle, Hydrafill, and Perlane. HA is typically derived from animal sources, such as rooster combs, or produced by bacterial strains. HA derived from animal sources is typically of higher molecular weight (4-6 MDa) than bacterial sources (1.5-2.5 MDa) due to the longer polymeric chain length. The particle density is an important factor in accelerating microparticles for skin penetration into the dermis, therefore in other embodiments, HA with the appropriate molecular weight is used.

In other embodiments, dextran microparticles, which are available as Sephadex are also used in microparticles. In further embodiments, a combination of Dextran and HA particles are used herein. Dextran is a biocompatible material that swells on hydration. In some embodiments, dextran is acquired in dry form as Sephadex™ and is available from GE Healthcare. In some embodiments, EAE Sephadex is utilized to increase swelling and also improve biocompatibility. Sephadex G 100 has dry power microspheres in a size range of about 40 μM to about 120 μM in diameter which swell to a diameter of about 100 to about 310 μM upon hydration. Superfine Sephadex G100 has a narrower dry particle range of about 20 μM to about 50 μM which swell to up to about 100 μM upon hydration. In general Sephadex G 100 will swell about 15 to about 20 ml/gram of dry powder. These Sephadex microspheres are substantially spherical in shape and have sufficient density and aerodynamic diameter for high velocity delivery to the skin. In some embodiments, the Sephadex is provided as sterile microspheres using sterile techniques (e.g. heat treatment, ethylene oxide, or radiation).

In yet further embodiments, dextran biodegradation occurs more slowly than HA providing a more sustained result. In yet another embodiments, the addition of dextran stimulates natural collagen formation. In further embodiments, other combinations of dermal fillers injected as substantially dry powders through high velocity are used as dermal fillers combining attributes from each filling agent.

Though HA fillers are derived from the polymer HA, they differ in regard to longevity. Natural HA has a half-life in tissue of about 1 to 2 days, undergoing aqueous dilution and enzyme degradation in the liver to the end products carbon dioxide and water. In some embodiments, the use of a dermal filler provides a longer residual tissue time. In other embodiments, cross-linking native HA provides greater stability (resistance to degradation by Haluronidase and free radicals), and thus increases longevity by creating larger macromolecules that retain the biocompatible and viscoelastic filling properties of natural hyalurons. As the degree of cross-linking increases, a liquid will first become a gel and then a solid. A higher degree of cross-linking results in a greater degree of resistance to degradation in the body. Too much cross-linking, though, requires a greater extrusion force to expel the product through the needle, resulting in significant tissue trauma. It can also potentially leave residual, free-floating “cross-linkers” unbound to the HA acid, which may be toxic. In some embodiments, substantially dry powder injection of HA microspheres (or microparticles) allow for more cross-linking and greater durability of the filler in the dermis since the issue of extrusion force and tissue trauma, eliminating the problems associated with needle and syringe devices. Further, it is easier to remove excess or residual cross linkers from cross-linked solid HA spheres, than it is to eliminate them from the gelatinous state, thereby resulting in a potentially safer product configuration.

In some embodiments, cross-linking is achieved by chemical reaction or through exposure to external energy sources such as ultraviolet radiation, infrared, conductive thermal, convective thermal, and ultrasonic sources. In some embodiments, commercially available HA used in dermal fillers are cross-linked with a linking group such as by way of example only 1,4-butanediol diglycidal ether (BDDE), and di-vinyl sulfone (DVS), which both react with hydroxyl sites on the HA chains. In other embodiments, other linking agents are suitable. Residual linking groups or cross-linking agents are removed as much as possible in the final products. The degree of cross-linking indicates the percentage of HA disaccharide monomer units that are bound to a cross-linker molecule. Thus, a dermal filler having a degree of cross-linking of 4% means that, on average, there are four cross-linker molecules for every 100 disaccharide monomeric units of HA. Optimal cross-linking may range from about 0.1% to about 30%.

In other embodiments, cross-linking with a linking group is used to control the swelling of the microparticles to optimize dermal filling by tailoring the degree of dermal volumizing to the needs of the patient. By way of example only, dextran microspheres, available as Sephadex, with varying degrees of cross-linking which determines the final relative microsphere diameter and volume upon hydration. In addition Sephadex is available as a diethylaminoethyl (DEAE) form which creates a positive charge at neutral pH. This allows the beads to swell even more upon hydration. In further embodiments, the tissue compatibility, bondability, and ability to penetrate tissue surfaces is increased. By way of example only, 1 gram of Sephadex G25 with 50 to 150 micrometer diameter particles will swell to about 4 ml to about 6 ml with deionized water; Sephadex G100 1 gram will swell to about 15 ml to about 20 ml with deionized water and Sephadex 200 will swell to about 30 ml to about 40 ml with deionized water. This swelling property is a function of the degree of cross-linking with G25 having more cross-linking than G100 and G200 having the least cross-linking. Thus in some embodiments, in an area of thinner skin with finer wrinkles is a method for treatment comprising using a product similar to G25. In other embodiments, for thicker skin with deeper wrinkles a method for treatment involves the use of G100 or G200 to provide greater volumizing effect. In further embodiments, these principles are applied to cross-linked HA microparticles to achieve the same treatment optimization.

In other embodiments, hyaluronic acid is also modified through esterification on one of its three available reactive groups (hydroxyl, carboxyl, acetamino) with various alkyl or benzyl groups or other moieties. In further embodiments, this reduces degradation and prolong residence time in the tissue. In yet other embodiments, esterified HA is formed into microparticles or microspheres. Esterification affects water absorption, therefore in some embodiments, esterification groups that preserve some water absorption properties are described herein.

In some embodiments, HA described herein is derived and substantially cross-linked in a manner typical for commercially available dermal fillers. In further embodiments, additional process steps are required to produce the desired substantially dry powder for high velocity transdermal injection. In other embodiments, following the cross-linking step the HA is formed into microparticles to produce HA of substantially spherical or ellipsoid shape of about 10 to about 100 micrometers mean aerodynamic diameter. Exemplary processes for microparticle formation include oil water single and double emulsification processes, microdroplet formation, affinity plate, and spray techniques. In further embodiments, the microparticles are then exposed to an aqueous solution of lidocaine which will associate into the microparticle. Following microparticle formation the particles are made partially or substantially dry. In yet further embodiments, suitable drying methods are used, for example spray-drying, free-drying, spray-freeze drying, air-drying, vacuum-assisted drying and the like. In yet other embodiments, the drying methods used are freeze-drying and spray-drying methods. In yet further embodiments, the drying process and the extent of water removal is used to produce particles of the optimal size and shape. In yet other embodiments, when lidocaine is incorporated it will remain trapped in the dried particle. Thus, in some embodiments, the process for fabricating suitable dermal fillers, such as for example, HA, for substantially dry powder injections includes the steps of: (1) substantially cross-linking the long chain polymer with a suitable linking group; (2) removing the substantially excess linking group; (3) forming the microparticle to a size range, hardness and density suitable to cross the stratum corneum and into the dermis in a partially or substantially dry form; and (4) drying the microparticles to a partially or substantially anhydrous state. In yet another embodiment, the incorporation of an additional agent such as lidocaine into the particle is achieved by suspending the particles in a substantially aqueous solution of lidocaine.

In other embodiments, the dermal filler compositions described herein are powders having HA or dextran (e.g. Sephadex or DEAE Sephadex) or other dermal filler material containing particles of a size appropriate for high-velocity transdermal delivery to a subject across the stratum corneum. In other embodiments, the powder is flowable. In yet other embodiments, the mean mass aerodynamic diameter of the particles forming the flowable powder range from about 0.1 μM to about 250 μM. In other embodiments, the particles forming the flowable powder is in the range of about 1 μM to about 200 μM; about 5 μM to about 150 μM; about 10 μM to about 125 μM; about 20 μM to about 100 μM; about 30 μM to about 90 μM; about 40 μM to about 80 μM. In other embodiments, less than about 75 μM. In other embodiments, the particles are in the range of about 40 μM to about 75 μM. In some embodiments, the particles of the powder have an envelope density of from about 0.1 to about 25 g/cm³. In other embodiments, the powder particles have an envelope density of from about 0.2 to about 10 g/cm³. In yet other embodiments, from about 0.5 to about 5 g/cm³; about 0.75 to about 2.0 g/cm³. In other embodiments, from about 0.8 to about 1.5 g/cm³. In some other embodiments, while the shape of the individual particles vary when viewed under a microscope, the shape is approximately spheroidal, but in further embodiments are elliptical, irregular in shape and/or toroidal. In some other embodiments, HA microparticles or filler materials such as dextran or Sephadex lend themselves to forming nearly spherical particles, which have regular or irregular surfaces. In yet other embodiments, these filler materials also lend themselves to forming particles of a uniform density having the active agent associated with the hydrogel particle by absorption throughout the particle or simply by association with the hydrogel particle surface. In yet other embodiments, each particle in the powder has a mean mass aerodynamic diameter of about 10 μM to about 100 μM.

### Cross-Linked Dermal Fillers

Provided herein are substantially cross-linked dermal fillers wherein the dermal filler comprises hyaluronic acid, hyaluronic acid derivatives or mixtures thereof, alginic acid, alginic acid derivatives or mixtures thereof which are substantially cross-linked via a combination of covalent and ionic bonds. In one aspect is a dermal filler composition comprising a substantially cross-linked composition of alginic acid, or a derivative of alginic acid or mixtures thereof or a pharmaceutically acceptable salt thereof hyaluronic acid, or a derivative of hyaluronic acid or mixture thereof and at least one Ca²⁺ ion.

In one embodiment is a dermal filler composition wherein the pharmaceutically acceptable salt of alginic acid, derivative of alginic acid or mixture thereof is selected from ammonium, calcium, potassium, or sodium salt. In another embodiment is a dermal filler composition wherein the pharmaceutically acceptable salt of alginic acid, derivative of alginic acid or mixture thereof is calcium salt. In yet another embodiment is a dermal filler composition wherein at least one hyaluronic acid, derivative of hyaluronic acid or mixture thereof is covalently attached to another hyaluronic acid, derivative of hyaluronic acid or mixture thereof. In a further embodiment is a dermal filler composition wherein at least one hyaluronic acid, derivative of hyaluronic acid or mixture thereof is covalently attached to at least one alginic acid, derivative of alginic acid, or mixture thereof via an ether linker. In yet a further embodiment is a dermal filler composition wherein at least one alginic acid, derivative of alginic acid or mixture thereof is covalently attached (substantially cross linked) to another alginic acid, derivative of alginic acid, or mixture thereof via a linker group such as a group providing an ether linker. In one embodiment is a dermal filler composition wherein the linking group is selected from a group consisting of BDDGE, polyethylene glycol, polypropylene glycol, tetraethylene glycol dimethacrylate, N,N′-methylenebisacrylamide, 2,2′-(2,2′-oxybis(ethane-2,1-diyl))dioxirane, 2,2′-(oxybis(methylene)bis(oxy)bis(methylene))dioxirane or derivatives thereof. In another embodiment is a dermal filler composition wherein alginic acid, derivative of alginic acid, or mixture thereof and hyaluronic acid, derivative of hyaluronic acid, or mixture thereof is in about a 1:5 to about a 5:1 ratio of alginic acid, derivative of alginic acid, or mixture thereof to hyaluronic acid, derivative of hyaluronic acid or mixture thereof. In another embodiment, the mixture is in about a 1:4 ratio; about a 1:3 ratio; about a 1:2 ratio; about a 1:1 ratio; about a 2:1 ratio; about a 3:1 ratio; about a 4:1 ratio; or about a 5:1 ratio of alginic acid, derivative of alginic acid, or mixture thereof to hyaluronic acid, derivative of hyaluronic acid or mixture thereof.

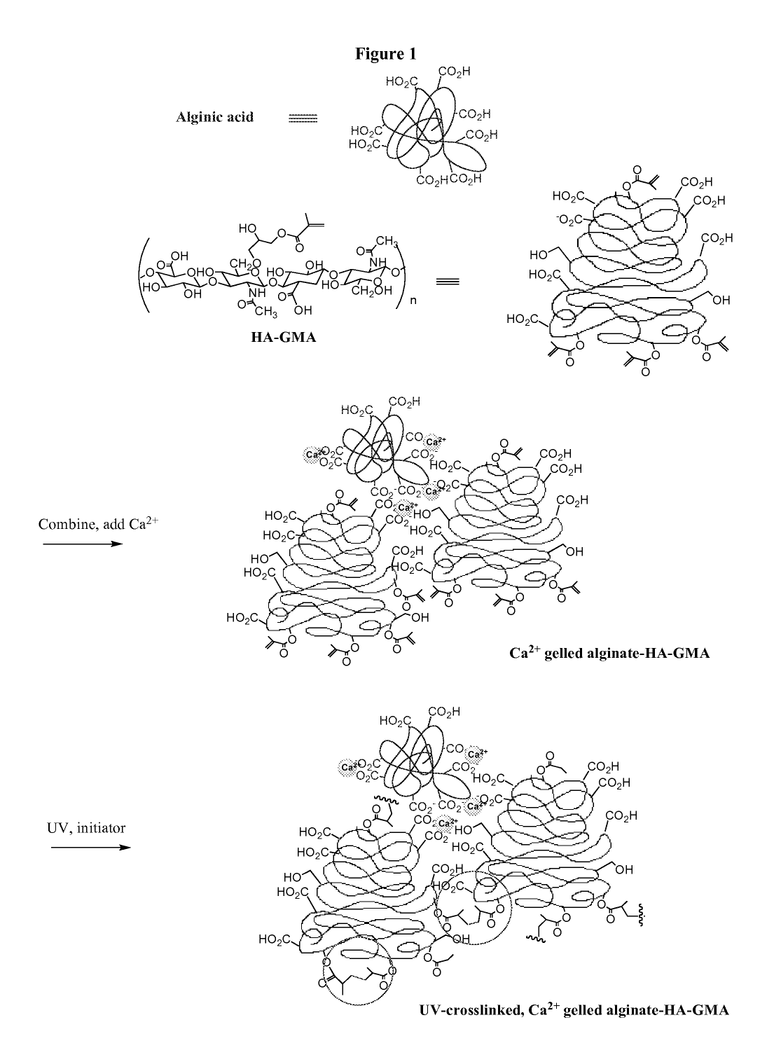

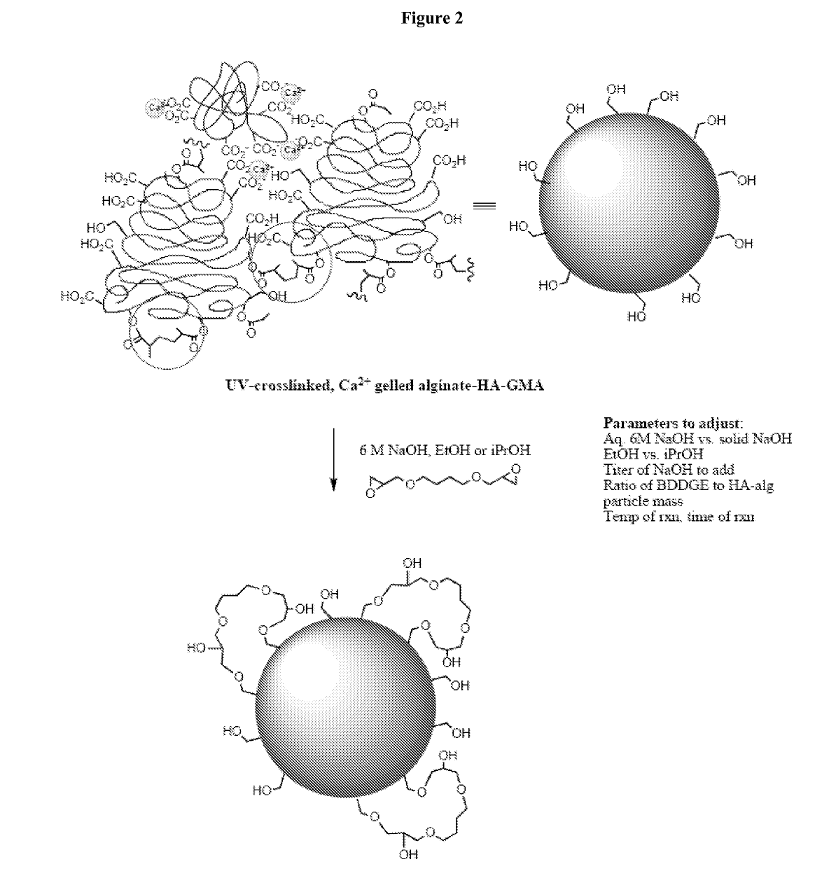

In one embodiment is a method of manufacturing a dermal filler substantially cross-linked composition as shown for example in FIGS. 1 and 2. By way of example only, alginic acid or methacrylated alginic acid and HA-GMA is combined and added to a solution containing Ca²⁺ ions. The solution containing Ca²⁺ ions assists in the formation of substantially spherical gelled Ca²⁺ alginate-HA-GMA microspheres. The use of Ca²⁺ ions in the formation of the microspheres described herein provides in some embodiments, an ionic cross-link between the alginic acid or methacrylated alginic acid and HA-GMA and enables formation of substantially spherical microspheres. An external energy source such as ultra-violet radiation in the presence of a photo initiator such as by way of example only, acetophenone is used to substantially cross-link the Ca²⁺ gelled alginate-HA-GMA microspheres. Suitable photo initiators such as benzophenone, diphenoxy benzophenone, halogenated and amino functional benzophenones, fluorenone derivatives, anthraquinone derivatives, zanthone derivatives, thioxanthone derivatives, camphorquinone, and benzil are also used herein.

A linking agent, such as for example, BDDGE is added to the UV crosslinked Ca²⁺ gelled alginate-HA-GMA microsphere after treatment of the UV crosslinked Ca²⁺ gelled alginate-HA-GMA microspheres with a suitable base, such as for example, 6 M NaOH, in either EtOH or iPrOH as a solvent. Suitable solvents for cross-linking are also used herein. Bases suitable to activate the primary OH groups of the HA are used herein, such as for example, lithium hydroxide, potassium hydroxide, calcium hydroxide, and barium hydroxide are also used herein.

Other suitable linking agents such as polyethylene glycol or derivatives of polyethylene glycol or molecules which provide an ether linker are also used herein. Linking agents which provide a cross-link having ester bonds, carbonate bonds, and carbamate bonds are also used herein in addition to the linking agents providing a cross-link having an ether bond.

In further embodiments, the microspheres are coated or compose an agent such as an anti-inflammatory agent, an anesthetic agent, such as by way of example only, lidocaine; dermal growth factors or fibroblast growth factors, including but not limited to proteins, steroids, cytokines, or hormones, a paralytic, such as by way of example only, clostridium botulinum toxin using the methods described above.

In another aspect is a method of manufacturing a dermal filler substantially cross-linked composition comprising:
(i) combining a derivative of alginic acid, or mixture of alginic acid/derivative of alginic acid or a pharmaceutically acceptable salt thereof; with a derivative of hyaluronic acid, or mixture of hyaluronic acid/derivative of hyaluronic acid; and Ca²⁺ ions;
(ii) providing an external energy source to form a first cross-linked combination;
(iii) adding a base to the first cross-linked combination to provide a basic form of the first cross-linked combination; and
(iv) adding a linking agent to the basic form of the first cross-linked combination to provide a substantially cross-linked composition.

In one embodiment is a method of manufacturing a dermal filler substantially cross-linked composition wherein the base is NaOH. In one embodiment, 6 M NaOH is used. In another embodiment, solid NaOH is used. In yet a further embodiment, EtOH or iPrOH is used as the solvent. In a further embodiment the linking agent is selected from 1,4-butanediol diglycidyl ether. Other suitable linking agents providing an ether linker, such as for example, polyethylene glycol or derivatives of polyethylene glycol are also used herein. In yet a further embodiment is a method of manufacturing a dermal filler substantially cross-linked composition further comprising purifying the substantially cross-linked composition. In yet another embodiment the external energy source comprises the use of ultraviolet radiation, infrared, conductive thermal, convective thermal, and ultrasonic sources. In a further embodiment the external energy source comprises the use of ultraviolet irradiation. In yet a further embodiment the use of ultraviolet irradiation is in the presence of a photosensitizer. In another embodiment, the photosensitizer is acetophenone. In one embodiment the use of ultraviolet irradiation is for a period of about 1 second to about 10 minutes. In another embodiment, the ultraviolet radiation is for a period of about 30 seconds to about 5 minutes; about 1 minute to about 4 minutes; about 2 minutes to about 3 minutes.
Also presented herein is a dermal filler substantially cross-linked composition prepared by the process comprising:
(i) combining a derivative of alginic acid, or a mixture of derivative of alginic acid/alginic acid or a pharmaceutically acceptable salt thereof; with methacrylate hyaluronic acid, or mixture of methacrylate hyaluronic acid/hyaluronic acid and Ca²⁺ ions;
(ii) irradiating the combination;
(iii) adding a base to the irradiated combination to provide a basic form of the irradiated combination; and
(iv) adding a linking agent to the basic form of the irradiated combination to provide a substantially cross-linked composition.

The cross-linking compositions and methods for preparing these compositions are not limited to alginic acid, derivatives of alginic acid, or mixtures thereof, and hyaluronic acid, derivatives of hyaluronic acid, or mixtures thereof but apply to other types of dermal filler materials such as for example, dextran, polymethacrylate, agarose, collagen, hydroxyapatite, polymethylmethacrylate, carboxymethyl cellulose, and their derivatives using the methods described herein.

In one embodiment is a process for providing substantially cross-linked hyaluronic acid as a dermal filler in a form suitable for delivery. In one embodiment, the process is applied to other polymers suitable for dermal filling.

### Dry Powder Injection Device

In some embodiments, the dermal filler compositions are delivered to the skin by high velocity delivery. In some embodiments, into skin target sites (epidermis, dermis, subdermis, or subcutis) using the energy of a transient helium gas jet at a predetermined area of skin or. In some embodiments, a “predetermined area” is the area of intact living skin. That area is usually in the range of about 0.3 cm² to about 10 cm². However, transdermal particle delivery velocities that are used to transfer drug materials into the target area where the composition is administered may vary significantly, depending on device configuration, dose, and the like. Injection velocities generally range from about 100 to about 3,000 m/sec such as from about 200 to about 2000 m/sec.

In some embodiments, the devices described herein are referred to as needleless syringe devices and representative of these devices are the dermal PowderJect® needleless syringe device needleless syringe device (PowderJect Technologies Limited, Oxford, UK, Zingo Devices Anesiva, Inc.). By using these devices, an effective amount of dermal filling agent, in some embodiments HA, agent is delivered to the skin or subcutis. An effective amount is that amount needed to give the desired volumizing effect to the skin to correct skin surface defects such as wrinkles, scars, or dermal thinning. In other embodiments, this amount varies with the area and defect to be corrected and is, in other embodiments measured clinically through photography and skin surface topography measurements.

Needleless syringe devices for delivering particles were first described in U.S. Pat. No. 5,630,796 (Bellhouse et al.), incorporated herein by reference to the extent relevant. Although a number of specific device configurations are now available, such devices are typically provided as a pen-shaped instrument containing, in linear order moving from top to bottom, a gas cylinder, a particle cassette or package, and a supersonic nozzle with an associated silencer medium. An appropriate powder (in the present case, a powder comprising the hydrogel particles) is provided within a suitable container, e.g., a cassette formed by two rupturable polymer membranes that are heat-sealed to a washer-shaped spacer to form a self-contained sealed unit. Membrane materials in other embodiments are selected to achieve a specific mode of opening and burst pressure that dictate the conditions at which the supersonic flow is initiated. In operation, the device is actuated to release the compressed gas from the cylinder into an expansion chamber within the device. The released gas contacts the particle cassette and, when sufficient pressure is built up, suddenly breaches the cassette membranes sweeping the particles into the supersonic nozzle for subsequent delivery. The nozzle is designed to achieve a specific gas velocity and flow pattern to deliver a quantity of particles to a target surface of predefined area. The silencer is used to attenuate the noise produced by the membrane rupture. The silencer in other embodiments also takes the shape of the desired pattern for delivery of dermal filler microparticle.

In some other embodiments, is the delivery of particles described herein over a region of skin in a particular pattern. By way of example only, a crescent shaped pattern is useful for treating skin under and around the eyes or corners of the mouth. A linear or circular or square pattern in other embodiments is suitable for the forehead and cheeks. This can accomplished in several ways but not limited to the methods described herein. First, in some embodiments, the nozzle and outflow cone is designed to deliver the powder over a predetermined pattern on the skin. In other embodiments, the outlet of the cone/silencer is partially masked with a material that absorbs or stops some particles prior to skin delivery. In further embodiments, the skin has an opening in the predetermined pattern and allowing particles to pass through according to the predetermined pattern. In another embodiment a microparticle absorbing tape is placed on the patients face to cover areas that are to be shielded and to allow patterning of skin delivery.

Another dry powder needleless injection device for delivering dermal filler particles is described in International Publication No. WO 96/20022, and is incorporated by reference to the extent relevant. This delivery system also uses the energy of a compressed gas source to accelerate and deliver powdered compositions; further it uses a shock wave instead of gas flow to accelerate the particles. More particularly, an instantaneous pressure rise provided by a shock wave generated behind a flexible dome strikes the back of the dome, causing a sudden eversion of the flexible dome in the direction of a target surface. This sudden eversion catapults a powdered dermal filler composition (which is located on the outside of the dome) at a sufficient velocity, thus momentum, to penetrate target tissue, e.g. skin. The powdered composition is released at the point of full dome eversion. The dome also serves to completely contain the high-pressure gas flow which therefore does not come into contact with the tissue. Because the gas is not released during this delivery operation, the system is inherently quiet. A preferred needleless dry powder injection device would allow multiple doses to be delivered over several regions during a single use on a patient. In yet a further aspect, single unit dosages or multidose containers, in which the dermal filler particles described herein are packaged prior to use, and in some embodiments comprise a hermetically sealed container enclosing a suitable amount of the particles that make up a suitable dose. The particle compositions in other embodiments, are packaged as a sterile formulation, and the hermetically sealed container is designed to preserve sterility of the formulation until use in the methods described herein. In other embodiments, the containers are adapted for direct use in the above-referenced needleless syringe systems.

In some embodiments, delivery of dermal filler particles from the above-referenced needleless syringe systems are used with particles having an approximate size ranging from about 0.1 to about 250 μM, in other embodiments, ranging from about 1 to about 100 μM; about 5 μM to about 90 μM; about 10 μM to about 75 μM; about 20 μM to about 50 μM. In further embodiments, particles larger than about 250 μM are also delivered from the devices, with the upper limitation being the point at which the size of the particles would cause untoward damage to cells at the target surface. The actual distance which the delivered particles will penetrate a target surface depends upon particle size (e.g., the nominal particle diameter assuming a roughly spherical particle geometry), particle density, the impact velocity at which the particle impacts the target surface, and the density, surface tension, and kinematic viscosity of the targeted skin tissue. In this regard, in some embodiments, particle densities for use in needleless injection generally range between about 0.1 and about 25 g/cm³; in other embodiments between about 0.9 and 1.5 g/cm³, and in yet other embodiments injection velocities range between about 100 and 3,000 m/sec. With appropriate gas pressure, particles having an average diameter of about 10 μM to about 80 μM are accelerated through the nozzle at velocities approaching the supersonic speeds of a driving gas flow.

### Treatment Methods

In one aspect is a method for correcting or modifying skin defects comprising providing substantially or partially dry microparticles of dermal filler; and delivering the substantially or partially dry microparticles to the dermis wherein the volume of the dermis is increased. In a further embodiment is a method for contouring facial features comprising administering substantially or partially dry microparticles of dermal filler to a subject in need.

In one embodiment is a method for correcting or modifying skin defects wherein the dermal filler comprises a material selected from hyaluronic acid, dextran, polymethacrylate, agarose, collagen, hydroxyapatite, polymethylmethacrylate, and carboxymethyl cellulose. In another embodiment the dermal filler comprises hyaluronic acid. In a further embodiment the hyaluronic acid is substantially cross-linked. In yet another embodiment the dermal filler comprises dextran. In a further embodiment dextran is substantially cross-linked. In one embodiment delivering the substantially or partially dry microparticles of dermal filler to the dermis comprises accelerating the substantially or partially dry microparticles at a velocity sufficient to penetrate the skin.

In another aspect is a method for delivering a dermal filler comprising delivering at a high velocity substantially or partially dry microparticles of dermal filler through the skin and into the dermis using a substantially dry powder needleless injection device to correct or modify skin defects such as for example, skin wrinkles.

In one embodiment is a method for delivering a dermal filler wherein the substantially or partially dry microparticles of dermal filler comprises a material selected from hyaluronic acid, dextran, polymethacrylate, agarose, collagen, hydroxyapatite, polymethylmethacrylate, and carboxymethyl cellulose. In another embodiment the dermal filler comprises hyaluronic acid. In yet another embodiment hyaluronic acid is substantially cross-linked. In a further embodiment the dermal filler comprises dextran. In yet a further embodiment dextran is substantially cross-linked. In another embodiment the substantially or partially dry microparticles further comprises or are coated with an anesthetic agent. In yet another embodiment the anesthetic agent is lidocaine. In one embodiment the substantially or partially dry microparticles further comprise or are coated with an anti-inflammatory agent. Anti-inflammatory agents include glucocorticosteroids as well as NSAIDs. In another embodiment the substantially or partially dry microparticles further comprise or are coated with dermal growth factors or fibroblast growth factors, including but not limited to proteins, steroids, cytokines, or hormones. In a further embodiment the substantially or partially dry microparticles further comprise or are coated with a paralytic. In yet a further embodiment the paralytic is clostridium botulinum toxin. In another embodiment the substantially or partially dry microparticles are coated with a biocompatible material having a sufficient high surface hardness wherein the penetration properties of the microparticles are enhanced. In one embodiment is a method for providing volume for skin contouring, skin correction, and surface defect correction comprising administering a dermal filler composition described herein. In one embodiment, the skin defect is a wrinkle. In another embodiment, the skin defect is a scar. In another embodiment is a method for reducing the pain associated with needle delivery devices comprising administering a dermal filler composition described herein via a needless injection device. In another embodiment is a method of treating broader dermal areas which are difficult to access using a needle based delivery device comprising administering a dermal filler described herein. In one embodiment is a method for providing a more uniform and controlled delivery of a dermal filler described herein to the dermis of the skin. In yet another embodiment is a method for delivering a dermal filler described herein in a unique coverage area shape, such as but not limited to, periorbital crescents, acne scars, lip features comprising administering a dermal filler using custom nozzle shapes and designs. In yet another embodiment is a method for treating patient discomfort and pain for patients undergoing dermal filling procedures.

In a further embodiment is a method of delivering substantially or partially dry microparticles comprising a hydrophilic dermal filler agent, such as by way of example only, hyaluronic acid or carboxymethyl cellulose) uniformly to the dermis of the skin thereby allowing the microparticles to swell by absorbing moisture from the surrounding tissue to provide a volumizing effect. In one embodiment, the volumizing effect corrects or modifies skin surface defects, such as for example, skin wrinkles and adds volume or the like.

In another embodiment cross-linked hyaluronic microparticles are delivered to the same area of skin. HA microparticles have similar density and size as Sephadex particles described above. In some embodiments, the HA microparticles absorb more water and swell to a larger size than the Sephadex depending on the degree of cross-linking and chain length and in other embodiments, swell from about 100 ml to about 1000 ml per gram of substantially dry powder. In some embodiments, particle sizes in the smaller range of about 20 μM to about 50 μM are used. In other embodiments, particle sizes from about 0.1 μM to about 100 μM are suitable. In further embodiments, the particle diameters increase by about 2 to about 10 times upon hydration. In some embodiments, when particles of 25 micrometers are delivered that swell to about 150 μM upon hydration and rehydration yields about 100 ml/gram of substantially dry powder, approximately 0.2 to about 20 mg of HA are utilized to achieve the same range of volume increase as the Sephadex microparticles.

Dermal filling with currently available needle and syringe techniques employ 0.5 ml to 2.0 ml of hydrated cross-linked HA to a similar region. Thus a substantially dry powder injection of HA or Sephadex microparticles in some embodiments achieve the same degree of dermal filling by delivery of a very small volume of initially dry powder. Further the current treatment using substantially dry powder will more uniformly disperse the HA throughout the treatment area and not require multiple needle repositionings. Because the initial volume of drug injected is much smaller the treatment associated pain is expected to be diminished. Slow tissue expansion, such as that used in plastic surgery to provide excess skin for grafting, is known to be less painful than sudden expansion as the tissue is able to accommodate increased volume over a longer time. Similarly the slower swelling of the dry particle is likely to produce less discomfort for the same resultant volume increase.

In one embodiment is the use of a substantially dry powder biolistic injection of dermal filling material wherein the pain associated with needlestick in tissue is reduced relative to traditional gel filled syringe injection techniques.

In another embodiment is the treatment of broader coverage areas with dermal filling material wherein the treatment to areas that may be difficult and time consuming to cover with existing needle syringe based gel tunnel injection techniques.

In a further embodiment is a method of providing more uniform and controlled delivery of dermal filler to the dermis of the skin.

In yet another embodiment is a method of delivering unique coverage area shapes, such as but not limited to, periorbital crescents, acne scars, lip features using custom nozzle shapes and designs.

In yet a further embodiment is a method of reducing patient discomfort and pain for patients desiring to undergo dermal filling procedures comprising administering the dermal fillers described herein using a dry powder biolistic injection method.

### Injectable Volumizing Compositions

In some embodiments, filler materials are also used to increase volume of the face (e.g. cheeks) or the body (e.g. breasts). In some embodiments, about 1 to about 10 ml of current HA dermal filler is used to provide volume to the face. To achieve a large end volume result, in one embodiment an anhydrous cross-linked microparticles of HA is suspended in a non-aqueous pharmaceutically acceptable liquid carrier. There are many non-aqueous liquid carriers available that are biocompatible and generally regarded as safe. Examples include propylene glycol and polyethylene glycol. In some embodiments, about 100 mgs to about 1 gram of anhydrous HA microparticles as described above are suspended in about 1 mL of non-aqueous liquid carrier. In one embodiment, injection of the 1 ml volume suspension yields a volumizing effect of about 100 ml to about 1000 upon hydration of the particles during absorption of the non-aqueous liquid carrier. Thus, in one embodiment, for a single injection of about 1 ml, approximately 100 to approximately 1000 times volumizing effect is achieved. In contrast currently available dermal fillers require injection of the actual 100 to 1000 ml of hydrated HA. In some embodiments, dextran microparticles are also used in a similar manner, or in combination with the HA.

While certain embodiments have been described, these embodiments have been presented by way of example only, and are not intended to limit the scope of the disclosure. The formulations, methods, and systems described herein may be embodied in a variety of other forms. Furthermore, various omissions, substitutions and changes in the form of the formulations, methods, and systems described herein may be made without departing from the spirit of this disclosure. The accompanying claims and their equivalents are intended to cover such forms or modifications.

### EXAMPLES

The following specific examples are to be construed as merely illustrative, and not limitative of the remainder of the disclosure in any way whatsoever. The examples described herein reference and provide non-limiting support to the various embodiments described in the preceding sections.

### Example 1

### Delivery to Human Skin In Vitro and Transepidermal Water Loss

For a composition performance test that more closely parallels eventual practical use, candidate particle compositions are injected into dermatomed, full thickness human abdomen skin samples. Replicate skin samples after injection are placed on modified Franz diffusion cells containing 32° C. water, physiologic saline or buffer. Additives such as surfactants are used to prevent binding to diffusion cell components. Two kinds of measurements are made to assess performance of the formulation in the skin.

To measure physical effects, i.e. the effect of particle injection on the barrier function of skin, the transepidermal water loss (TEWL) is measured. Measurement is performed at equilibrium (about 1 hour) using a Tewameter™ 210® (Courage & Khazaka, Koln, Ger) placed on the top of the diffusion cell cap that acts like a chimney about 12 mm. Larger particles and higher injection pressures generate proportionally higher TEWL values in vitro and is shown to correlate with results in vivo. Upon particle injection in vitro TEWL values increased from about 7 to about 27 (g/m^{2h}) depending on particle size and helium gas pressure. Helium injection without powder has no effect. In vivo, the skin barrier properties return rapidly to normal as indicated by the TEWL returning to pretreatment values in about 1 hour for most powder sizes. For the largest particles, 53-75.micron, skin samples show 50% recovery in an hour and full recovery by 24 hours.

### Example 2

A dry powder injection device with a cone diameter of 2 cm is applied to the lateral cheek. The dermal volume of the area of application is approximately 0.3 to 0.9 ml. 1 to 100 mg and 10 to 50 mg of Sephadex G 100 are delivered to the dermis over the 2 cm diameter application area. Upon hydration the Sephadex will increase in volume by 0.015 to 2.0 ml increasing dermal volume by approximately 5% to 200%.

The examples and embodiments described herein are for illustrative purposes only and various modifications or changes are included within the spirit and purview of this application and scope of the appended claims. All publications, patents, and patent applications cited herein are hereby incorporated by reference for all purposes.

What is claimed is:

1. A method for correcting or modifying skin defects comprising:
providing substantially or partially dry microparticles of dermal filler; and
delivering the substantially or partially dry microparticles to the dermis wherein the volume of the dermis is increased.

2. The method of claim 1 wherein the dermal filler comprises a material selected from hyaluronic acid, dextran, polymethacrylate, agarose, collagen, hydroxyapatite, polymethylmethacrylate, and carboxymethyl cellulose.

3. The method of claim 2 wherein the dermal filler comprises hyaluronic acid.

4. The method of claim 3 wherein the dermal filler is hyaluronic acid.

5. The method of claim 4 wherein hyaluronic acid is substantially cross-linked.

6. The method of claim 2 wherein the dermal filler comprises dextran.

7. The method of claim 6 wherein dextran is substantially cross-linked.

8. The method of claim 1 wherein delivering the substantially or partially dry microparticles of dermal filler to the dermis comprises accelerating the substantially or partially dry microparticles at a velocity sufficient to penetrate the skin.

9. The method of claim 1 wherein the skin defect is a wrinkle.

10. A dermal filler composition comprising a substantially or partially dry microparticle comprising a material selected from hyaluronic acid, dextran, polymethacrylate, agarose, collagen, hydroxyapatite, polymethylmethacrylate, and carboxymethyl cellulose.

11. The dermal filler composition of claim 10 wherein the material comprises hyaluronic acid.

12. The dermal filler composition of claim 11 wherein the material is hyaluronic acid.

13. The dermal filler composition of claim 12 wherein the hyaluronic acid is substantially cross-linked.

14. The dermal filler composition of claim 10 wherein the material comprises dextran.

15. The dermal filler composition of claim 14 wherein the material is dextran.

16. The dermal filler composition of claim 15 wherein dextran is substantially cross-linked.

17. The dermal filler composition of claim 10 further comprising a pharmaceutically acceptable carrier.

18. The dermal filler composition of claim 10 substantially free of a pharmaceutically acceptable carrier.

19. The method of claim 1 wherein the substantially or partially dry microparticles further comprises or are coated with an anesthetic agent.

20. The method of claim 19 wherein the anesthetic agent is lidocaine.

21. The method of claim 1 wherein the substantially or partially dry microparticles further comprise or are coated with an anti-inflammatory agent.

22. The method of claim 1 wherein the substantially or partially dry microparticles further comprise or are coated with dermal growth factors or fibroblast growth factors, including but not limited to proteins, steroids, cytokines, or hormones.

23. The method of claim 1 wherein the substantially or partially dry microparticles further comprise or are coated with a paralytic.

24. The method of claim 23 wherein the paralytic is clostridium botulinum toxin.

25. The method of claim 1 wherein the substantially or partially dry microparticles are coated with a biocompatible material having a sufficient high surface hardness wherein the penetration properties of the microparticles are enhanced.
